(19) United States
(12) Patent Application Publication
(10) Pub. No.: US 2021/0169110 A1
(43) Pub. Date: Jun. 10, 2021

(54) METHOD FOR EXTRACTING WASABI EXTRACT

(71) Applicant: WOOMTREE CO., LTD., Pocheon-si Gyeonggi-do (KR)

(72) Inventor: Yon Chan JEONG, Gimpo-si Gyeonggi-do (KR)

(21) Appl. No.: 16/770,293

(22) PCT Filed: Dec. 27, 2017
(86) PCT No.: PCT/KR2017/015526

§ 371 Date: Jun. 5, 2020

(30) Foreign Application Priority Data
Dec. 8, 2017 (KR) 10-2017-0168401

(51) Int. Cl. A23L 27/24; B01D 11/02; C07H 5/10

(52) U.S. Cl. CPC A23L 27/24; C07H 5/10; B01D 11/02

(57) ABSTRACT

A method for extracting a wasabi extract is characterized in that: 53 to 61 part by weight of a wasabi powder and 25 to 30 parts by weight of pure scotch whisky are mixed to produce a wasabi extract; and the wasabi extract is placed in an oak barrel and fermented for at least 48 hours, after which 14 to 17 parts by weight of a wasabi sinigrin extract that has collected in an upper layer portion is extracted therefrom. Further, when fermenting the wasabi extract to which undiluted whisky has been added inside the oak barrel, wasabi inside yeast cell walls formed in the upper layer portion is collected, and the method has the effect of maintaining the taste and aroma of the wasabi extract collected inside the yeast cell walls, that is, the sinigrin extract.

### TECHNICAL FIELD

The present invention relates to a method for extracting a wasabi extract and more particularly, to a method for extracting a wasabi extract in which when a wasabi extract to which undiluted whisky has been added is fermented inside an oak barrel, wasabi inside yeast cell walls formed in an upper layer portion is collected, and the taste and aroma of the wasabi extract collected inside the yeast cell walls, that is, a sinigrin extract are maintained.

### BACKGROUND ART

Wasabia koreana (Wasabia japonica, hereinafter referred to as “wasabi”) is an evergreen, perennial ripened semi-perennial plant belonging to the cruciferous family, and is known as a substance that may be used for anti-corruption, antibacterial and edible spices by containing sinigrin, allyl isothiocyanate, and vitamin C, glucose, KHSO4, etc. In order to use this, various attempts have been made, such as Patent No. 10-2005-0076893, in which a composition and food for preventing neurological diseases containing a wasabi extract were produced, and Patent No. 10-2011-035821, in which a natural edible antibacterial agent for food additives was produced through complex nano-particulation of a wasabi extract.

Currently, wasabi has been used by preparing the root of the western wasabi into dry powder, which has a disadvantage that it is difficult to maintain unique taste and aroma of wasabi and original taste and active ingredients thereof.

### DISCLOSURE

### Technical Problem

The present invention is invented to meet the needs of the prior arts described art, and an object of the present invention is to obtain sinigrin which is a taste and aroma ingredient and an active ingredient by collecting, filtering, and extracting wasabi raw materials in yeast cell walls.

### Technical Solution

A first embodiment of the present invention provides a method for extracting a wasabi extract, the method being characterized in that: 53 to 61 parts by weight of a wasabi powder and 25 to 30 parts by weight of pure scotch whisky are mixed to produce a wasabi extract, and the wasabi extract is placed in an oak barrel and fermented for at least 48 hours to extract 14 to 17 parts by weight of a wasabi sinigrin extract collected in an upper layer portion.

### Advantageous Effects

According to the method for extracting the wasabi extract of the present invention, there are effects that when a wasabi extract to which undiluted whisky has been added is fermented inside an oak barrel, wasabi inside yeast cell walls formed in an upper layer portion is collected, and the taste and aroma of the wasabi extract collected inside the yeast cell walls, that is, a sinigrin extract are maintained.

### Best Mode of the Invention

A first embodiment of the present invention provides a method for extracting a wasabi extract, the method being characterized in that: 53 to 61 parts by weight of a wasabi powder and 25 to 30 parts by weight of pure scotch whisky are mixed to produce a wasabi extract, and the wasabi extract is placed in an oak barrel and fermented for at least 48 hours to extract 14 to 17 parts by weight of a wasabi sinigrin extract collected in an upper layer portion.

### Mode of the Invention

Hereinafter, a preferred embodiment of the present invention will be described so as to be easily implemented by those skilled in the art. In describing the present invention, the detailed description of associated known functions or known constitutions will be omitted if it is determined that the detailed description unnecessarily makes the gist of the present invention unclear. In addition, certain features shown in the drawings are enlarged or reduced or simplified for ease of explanation, and although the components are not necessarily shown in proper proportions, those skilled in the art will easily understand these details.

Unless otherwise defined, all terms used herein including technological or scientific terms have the same meanings as those generally understood by those skilled in the art to which the present invention belongs. Terms which are defined in a generally used dictionary should be interpreted to have the same meaning as the meaning in the context of the related art, and are not interpreted as an ideal meaning or excessively formal meanings unless otherwise clearly defined in the present application.

Hereinafter, a preferable embodiment of the present invention will be described in detail. Moreover, in describing the present invention, the detailed description of related known configurations or functions will be omitted if it is determined that the detailed description makes the gist of the present invention unclear.

### Example

A sinigrin ingredient of wasabi meets oxygen and a thioglucosidase enzyme and chemically reacts with the oxygen and the thioglucosidase enzyme to discharge the vapor of an ally isothiocyanate ingredient. Allyl isothiocyanate, a main ingredient of spicy taste and aroma of wasabi, has antibacterial and antiseptic effects. The allyl isothiocyanate suppresses the growth of food poisoning causative bacteria, such as E. coli, Salmonella, 0-157, Vibrio, and Staphylococcus aureus.

The wasabi contains superoxide dismutase (SOD), an enzyme that removes active oxygen. The superoxide dismutase is known to act as an antioxidant defense mechanism in almost all cells exposed to oxygen.

The wasabi stimulates the stomach wall to awaken a central nervous system and promotes the secretion of digestive fluids, thereby helping to improve symptoms such as indigestion. In addition, an enzyme called beta amylase contained in the wasabi affects appetite enhancement. According to one study, it was confirmed that when a patient with poor appetite had a meal by adding 1 g of wasabi per day, the amount of meals increased due to increase in edible food.

At the International Chemical Conference on the Pacific Coast in 2000, a study result has been reported that mixtures that cause unique flavor of the wasabi can prevent tooth decay-causing bacteria from sticking to teeth.

Further, even in the research institute of a seasoning producing company in Japan, it was confirmed that the wasabi inhibits oral bacteria of forming acids that corrodes the tooth enamel.

In the wasabi, 41.g mg of vitamin C per 100 g is contained. The content of the vitamin C corresponds to about 10 times of apple (4.6 mg/100 g). There is no significant difference from lemon (53 mg/100 g) which comes to your mind when you think vitamin C.

Meanwhile, foods with strong spicy taste and aroma, such as wasabi may cause stomach problems such as heartburn and abdominal pain when the foods are consumed excessively.

The present invention is to obtain sinigrin which is a taste and aroma ingredient and an active ingredient by collecting, filtering, and extracting wasabi raw materials in yeast cell walls.

In general, in order to collect a wasabi aroma material, a method of re-collecting an enzyme and arabic gum or modified starch has been used, but a wasabi active ingredient is not naturally collected. Accordingly, in the present invention, the wasabi active ingredient is naturally collected in the yeast cell walls by applying a method for collecting a whiskey flavor in an oak barrel to obtain an extract composition for collecting the wasabi sinigrin active ingredient.

Currently, the wasabi has been used by making the root of the western wasabi into dry powder, which has a disadvantage that it is difficult to maintain unique taste and aroma of the wasabi and original taste and active ingredients.

Therefore, the present inventor improved a method of extracting wasabi in order to overcome the disadvantage and developed a preparing method in which when a sinigrin ingredient of wasabi having antibacterial and bactericidal effects meets oxygen and a thioglucosidase enzyme and then chemically reacts with the oxygen and the thioglucosidase enzyme, the sinigrin ingredient is collected in a process of becoming to an ally isothiocyanate ingredient, and then invented an extract composition containing a sinigrin active ingredient. That is, in the method for extracting the wasabi extract of the present invention, 53 to 61 parts by weight of a wasabi powder and 25 to 30 parts by weight of pure scotch whisky are mixed to produce a wasabi extract, and the wasabi extract is placed in an oak barrel and fermented for at least 48 hours to extract 14 to 17 parts by weight of a wasabi sinigrin extract collected in an upper layer portion.

### Experimental Example

A product used in an experiment was experimented as a refrigerated product. Analysis for ally isothiocyanate and wasabi sinigrin collected in an upper layer portion as an active ingredient was performed with active ingredient contents by using “Direct and simultaneous analysis of sinigrin and allyl isothiocyanate in mustard samples by High-Performance Liquid Chromatography, J. Agric. Food Chem. 2002” and analysis for vitamin B6 was performed by “2.2.2.16. Simultaneous analysis of vitamin B group, 2.2.2. Vitamins, 2.2. Micronutrient test method, 2. Food ingredient test method, 7. General test method in the Korean food standards codex”.

1. Content Results of Ally Isothiocyanate by Sample

TABLE 1

| Sample name | Content (mg/100 g) |
|---|---|
| Wasabi powder 55% | 1130.89 ± 46.34 |
| Wasabi extract 25% by scotch whiskey | 35.56 ± 0.65 |
| fermentation | |
| Wasabi extract 30% by scotch whiskey | 42.67 ± 1.77 |
| fermentation | |
| Wasabi sinigrin extract 14% | 16.10 ± 0.68 |
| Wasabi sinigrin extract 17% | 14.69 ± 1.20 |

2. Content Results of Sinigrin by Sample

TABLE 2

| Sample name | Content (mg/100 g) |
|---|---|
| Wasabi powder 55% | 252.37 ± 0.26 |
| Wasabi extract 25% by scotch whiskey | 474.47 ± 0.74 |
| fermentation | |
| Wasabi extract 30% by scotch whiskey | 1035.80 ± 7.10 |
| fermentation | |
| Wasabi sinigrin extract 14% | 5.43 ± 0.19 |
| Wasabi sinigrin extract 17% | 5.44 ± 0.29 |

As shown in [Table 2], it could be seen that a wasabi powder was mixed with a scotch whiskey solution and fermented, and as a result, the content of sinigrin was increased 2 times to 4 times.

3. Content Results of Vitamin B6 by Sample

TABLE 3

| Sample name | Content (mg/100 g) |
|---|---|
| Wasabi powder 55% | 10.14 ± 1.21 |
| Wasabi extract 25% by scotch whiskey | 2.40 ± 0.20 |
| fermentation | |
| Wasabi extract 30% by scotch whiskey | 4.00 ± 0.38 |
| fermentation | |
| Wasabi sinigrin extract 14% | 1.69 ± 0.12 |
| Wasabi sinigrin extract 17% | 1.96 ± 0.08 |

The wasabi sinigrin active ingredient can be used in various wasabi application products in the future and can be expected to be used for a functional ingredient of the active ingredient.

According to the present invention, the pure scotch whisky was mixed with the wasabi and fermented and extracted to collect wasabi sinigrin, and as a result, the content of sinigrin was significantly increased to maintain the unique aroma and taste of wasabi.

The wasabi sinigrin ingredient extracted by the extraction method as described above meets oxygen and a thioglucosidase enzyme and chemically reacts with the oxygen and the thioglucosidase enzyme to obtain an ally isothiocyanate ingredient.

In addition, an oak barrel was used to stably improve the unique taste and aroma of wasabi. The fermented wasabi extract in the oak barrel is collected in a hexagonal structure in the upper layer portion to minimize a portion where the taste and aroma of wasabi are lost.

According to the present invention, the reason why the taste and aroma of wasabi are maintained is that when fermenting the wasabi extract to which undiluted whisky has been added inside the oak barrel, wasabi inside yeast cell walls formed in the upper layer portion is collected to maintain the taste and aroma of the wasabi extract collected inside the yeast cell walls, that is, the sinigrin extract.

As described above, detailed embodiments have been described in the detailed description of the present invention, but it will be apparent that there is a possibility that the technology of the present invention will be easily modified and implemented by those skilled in the art and these modified embodiments will be included in the technical spirit disclosed in the appended claims of the present invention.

### INDUSTRIAL AVAILABILITY

According to the method for extracting the wasabi extract of the present invention, when a wasabi extract to which undiluted whisky has been added is fermented inside an oak barrel, wasabi inside yeast cell walls formed in an upper layer portion is collected, and the present invention has the effect of maintaining the taste and aroma of the wasabi extract collected inside the yeast cell walls, that is, the sinigrin extract.

1. A method for extracting a wasabi extract, wherein 53 to 61 parts by weight of a wasabi powder and 25 to 30 parts by weight of pure scotch whisky are mixed to produce a wasabi extract, and the wasabi extract is placed in an oak barrel and fermented for at least 48 hours to extract 14 to 17 parts by weight of a wasabi sinigrin extract collected in an upper layer portion.
